(19) United States
(12) Patent Application Publication
(10) Pub. No.: US 2015/0142009 A1
(43) Pub. Date: May 21, 2015

(54) STONE EXTRACTOR

(71) Applicant: Jiyong Ahn, Totowa, NJ (US)

(72) Inventor: Jiyong Ahn, Totowa, NJ (US)

(21) Appl. No.: 14/546,254

(22) Filed: Nov. 18, 2014

Related U.S. Application Data
(60) Provisional application No. 61/905,863, filed on Nov. 19, 2013.

(30) Foreign Application Priority Data
Jan. 2, 2014 (KR) 10-2014-0000088

(51) Int. Cl. A61B 17/221

(52) U.S. Cl. CPC A61B 17/221; A61B 2017/22035
USPC 606/127

(57) ABSTRACT

A general-purpose stone extractor is provided for removing gallstones, calculi or foreign bodies from the biliary tree, urinary tract, blood vessels and the like. The stone extractor is configured to enable catcher wires to be extracted and retracted from an outer sheath near to a stone by operating a push handle member, an elastic spring being installed between a proximal end of the outer sheath and the push handle member.

### CROSS-REFERENCE TO RELATED APPLICATION

This application claims priority to U.S. Provisional Patent Application No. 61/905,863, filed on Nov. 19, 2013, and Korean Patent Application No. 10-2014-0000088, filed on Jan. 2, 2014, which claims priority to the former U.S. Provisional Patent Application No. 61/905,863, under 35 U.S.C. 119, the entire contents of which are hereby incorporated by reference.

### BACKGROUND

1. Field of the Invention

The present invention relates to medical devices, and more particularly to stone extractors for removing stones in passages of various organs such as the biliary tree, urinary tract and the like.

2. Description of the Related Art

When stones are developed in passages of organs of the human body such as the biliary tree, urinary tract and the like, they block the normal flow and cause various diseases.

Although the methods for removing stones are different according to where stones are found and what symptoms appear, the stones are generally removed by a percutaneous extraction with a surgical x-ray apparatus such a c-cam or an imaging modality or by using an endoscope.

The percutaneous extraction is usually performed with a device consisted of a hollow lumen flexible sheath (i.e. a catheter) and a stone catching means positioned in the sheath, and the related prior arts are International Publication No. WO 1999/47054, Korean Patent No. 10-0124073 (Korean Patent Publication No. 10-1997-0009352), Korean Patent No. 10-0928706, etc. The prior arts related to using the endoscope are Korean Patent Publication No. 10-1998-0085829, etc.

International Publication No. WO 1999/47054 discloses catheter built-in wires having a wedge-shaped cross section and filling the interior of the catheter for maximally reducing the diameter of the catheter. Because there is no space between the built-in wires, it is difficult to insert a guide wire for guiding the wires to the location of a stone.

Korean Patent No. 10-0124073 discloses a stone catching means having a polygonal circular cone-shaped basket formed of a plurality of curved and fixed elastic wires. In order to open and close the entrance of the basket, it additionally needs a basket entrance opening and closing wire. Thus, the configuration of the stone catching means is complicated and it is difficult to easily remove the stone by controlling the basket entrance opening and closing wire.

Korean Patent No. 10-0928706 discloses a basket-shaped stone catching means configured with a plurality of elastic wires at a distal end and a control member at a proximal end for distally and proximally moving the basket. Because the basket must be tightly stretched by proximally moving the control member for catching the stone, it is uncomfortable during the operation. And because a vibration means and the like are installed on the internal side of the distal portion of an inserting tube (i.e., a catheter), there is a problem increasing the diameter of the inserting tube

Korean Patent Publication No. 10-1998-0085829 discloses a stone removing means having a distal end portion formed of a shape memory alloy to remove stones through the channel of an endoscope. But it has some problems. First, it is not substantially described on the material of the shape memory alloy, the condition for the transformation and the transformed shape for removing the stones. Second, during the transfer of the stone removing means into the body through the channel of the endoscope, because the stone removing means is already transformed into an original shape by a body temperature, it is difficult to catch the stones by the original shape. Third, although the distal end portion of the original shape could catch a stone, it is difficult to move the caught stone into the channel of the endoscope.

Furthermore, the described prior arts are restricted to the use of either endoscopic or percutaneous approach but not both operating conditions because of its configurations and it is difficult to remove foreign bodies from blood vessels.

### SUMMARY OF THE INVENTION

The present invention is contrived to solve the problems of the conventional technology and particularly, the objective is to provide a general-purpose stone extractor which enables removal of gallstones, calculi and foreign bodies not only from the biliary tree, urinary tract and the like, but also in an intravascular space.

To achieve the objective of the present invention, a stone extractor according to the present invention comprises: an inner core shaft formed of a single or plurality of strands, the inner core shaft having a hollow space in a longitudinal direction for inserting a guide wire; catcher wires connected to a distal end of the inner core shaft; a push handle member connected to a proximal end of the inner core shaft, the push handle member having a penetrating hole in the middle along a longitudinal direction; a flexible outer sheath configured to wholly wrap around the inner core shaft and the catcher wires and to extract and retract the catcher wires by operating the push handle member; and an elastic spring installed between a proximal end of the flexible outer sheath and the push handle member.

Here, the inner core shaft may be formed of a single strand having a penetrating hole in the middle along a longitudinal direction as the hollow space.

The single strand may be formed in one body with the push handle member.

The inner core shaft may be formed of a plurality of strands, the hollow space may be disposed between the plurality of strands, and each of the plurality of catcher wires may be connected to each of the plurality of strands in one-to-one correspondence.

The plurality of strands may be tied by one or more stabilizing members along a longitudinal direction.

Each of the plurality of strands may be formed in one body with each of the catcher wires

The plurality of strands may be consisted of 2˜8 strands.

The catcher wires may be made of shape memory alloy changing into an original shape at a predetermined temperature or super elastic alloy returning to an original shape by removing the restraining force of the outer sheath.

Each of the catcher wires may have an atraumatic distal end bending or curling toward the inside of the catcher wires.

The push handle member may have one of asymmetric T, symmetric T, asymmetric H and symmetric H-shaped handles.

The push handle member may further have a fixing means configured to be fixed to a proximal end of the sheath while the elastic spring is pressed down.

The push handle member may further comprise a supporting shaft to be wrapped around by the elastic spring, the supporting shaft being disposed between the handle and the inner core shaft. The fixing means may be consisted of a first guide groove formed at the supporting shaft in a longitudinal direction and a second guide groove formed at a proximal portion of the supporting shaft in a circumference direction, the second guide groove being vertically connected to the first guide groove. The proximal end of the sheath may have a groove inserting protrusion configured to move along the first and second guide grooves.

The fixing means may be consisted of a rack protrusion connected to the handle, and the proximal end of the outer sheath may have a concave grooves formed inside to be inserted by the rack protrusion.

The outer sheath may be consisted of a body tube wrapping around the inner core shaft and the catcher wires and an entrance tube having a flange connected to a proximal portion of the body tube, the fixing means may be consisted of one or more hook wires, and one end of each of the hook wires may be rotatably connected to the handle and the other end of each of the hook wires may have a bent shape to be hooked up to the flange of the outer sheath.

A stone extractor according to the present invention can extract and retract the catcher wires from the outer sheath by operating the push handle member because of the elastic spring installed between a proximal end of the flexible outer sheath and the push handle member. Thus, it is easy to make the outer sheath be approached near to and to catch a stone by the restoring force of the elastic spring. It is also possible to move the stone in a caught state by the restoring force of the elastic spring and to be efficiently removed.

And when each of the strands of an inner core shaft is formed in one body with each of catcher wires, a hollow space for inserting a guide wire can be obtained between the strands and the diameter of the outer sheath can be minimized. Thus, the present invention can be universally used to remove gallstones or calculi by using the endoscope or the percutaneous extraction, foreign bodies from blood vessels and the like.

Moreover, because a predetermined fixing means is further formed at a push handle member, it is possible to prevent from smashing and missing the stone caught by the restoring force of the elastic spring.

### BRIEF DESCRIPTION OF THE DRAWINGS

FIGS. 1(a)-1(c) are side elevational views, partly in section, showing a structure and an operation of a stone extractor according to an embodiment of the present invention.

FIGS. 2(a) to 2(d) are cross sectional views along line A-A, B-B and C-C in FIG. 1, respectively, and FIG. 2(d) is a cross-sectional view similar to FIG. 2(b), but showing another embodiment having an inner core shaft made of five strands.

FIG. 3(a) is a cross sectional view of an embodiment showing a push handle member having a penetrating hole in the middle along a longitudinal direction as shown in FIG. 1, and FIG. 3(b) is another embodiment of the present invention and shows a cross sectional view corresponding to FIG. 3(a) when a push handle member further comprises a cylindrical supporting shaft.

FIG. 4(a) is a perspective view of an embodiment showing an inner core shaft formed of a plurality of strands. FIG. 4(b) is a perspective view of an embodiment showing an inner core shaft formed of a single strand.

FIGS. 5(a)-5(f) are side elevational views showing exemplar shapes of catcher wires according to the present invention.

FIGS. 6(a)-6(c) are side elevational views showing exemplar shapes of a push handle member according to the present invention.

FIGS. 7(a), 7(b), 8(a), 8(b), 9(a) and 9(b) are partial side elevational views showing mechanisms for fixing a push handle member to an outer sheath overcoming the recovering force of an elastic spring by a fixing means according to the present invention.

FIG. 10(a) is an illustration and partially enlarged views showing an example for removing a stone in a urinary tract by a stone extractor according to the present invention and FIGS. 10(b) and 10(c) are enlarged views of the region A in FIG. 10(a).

In these drawings, the following reference numbers are used throughout: reference number 10 indicates a push handle member, 12 a handle, 14 a supporting shaft, 16 a penetrating hole, 18 a cylindrical supporting shaft, 20 an inner core shaft, 22 a catcher wire, 24 a distal end of a catcher wire, 30 an outer sheath, 32 a flange, 40 an elastic spring and 50 a stabilizing member.

### DETAILED DESCRIPTIONS

Detailed descriptions of preferred embodiments of the present invention are provided below with reference to accompanying drawings to be easily understood by those skilled in the art having ordinary knowledge in the technical field of the present invention. However, the present invention is not to be limited to the disclosed embodiments below, it is intended to be embodied to various applications based on the disclosed embodiments.

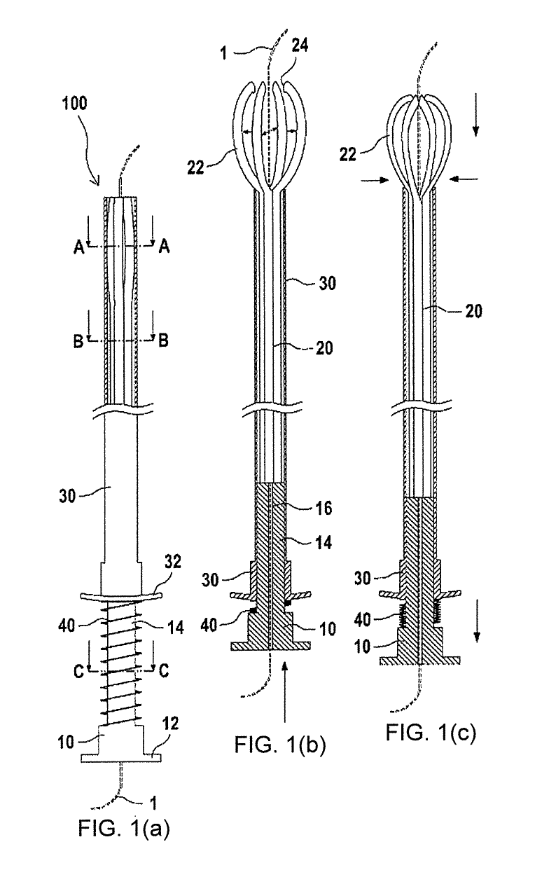

A stone extractor 100 according to the present invention basically, as commonly shown in FIGS. 1 to 4, comprises: an inner core shaft 20 formed of a single or plurality of strands, the inner core shaft 20 having a hollow space in a longitudinal direction for inserting a guide wire 1; catcher wires 22 connected to a distal end of the inner core shaft 20; a push handle member 10 connected to a proximal end of the inner core shaft 20, the push handle member 10 having a penetrating hole 16 in the middle along a longitudinal direction; a flexible outer sheath 30 configured to wholly wrap around the inner core shaft 20 and the catcher wires 22 and to extract and retract the catcher wires 22 by operating the push handle member 10; and an elastic spring 40 installed between the proximal end of the flexible outer sheath 30 and the push handle member 10.

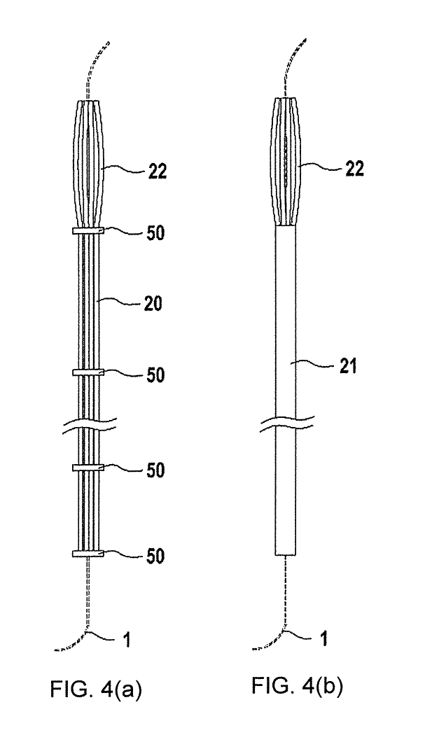

As concrete embodiments, the inner core shaft 20, as shown in FIG. 4(b), may be formed of a single strand 21 having a penetrating hole (not shown) in the middle along a longitudinal direction as the hollow space for a guide wire 1. In this case, the single strand 21 can be separately formed from the push handle member 10. But it is preferable that the single strand 21 is formed in one body with the push handle member 10 because it doesn't need any connecting means between them. Namely, in FIG. 3(a), a supporting shaft 14 having a penetrating hole 16 in the middle along a longitudinal direction is preferably extended to a connecting region of the catcher wires 22 as the single strand 21.

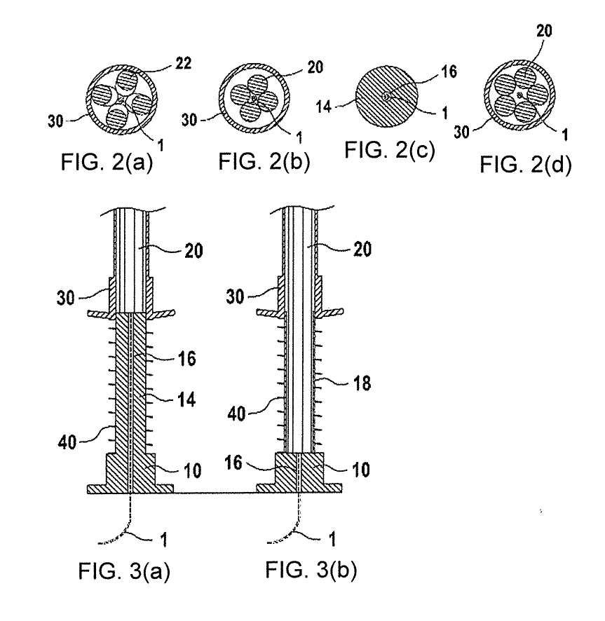

In another embodiment, the inner core shaft 20 may be formed of a plurality of strands as shown in FIG. 4(a), the hollow space for the guide wire 1 may be formed between the plurality of strands 20 as shown in FIG. 2(b), and each of the plurality of catcher wires 22 may be connected to each of the plurality of strands 20 in one-to-one correspondence.

By the above mentioned embodiment, it has a merit that there is no need to separately form a penetrating hole for a guide wire 1 along a longitudinal direction at the inside of the inner core shaft 20.

And it is preferable that the plurality of strands 20 are tied by one or more stabilizing members 50 along a longitudinal direction as shown in FIG. 4(a). Because it enables the stabilizing members 50 to prevent the strands from bending in the outer sheath 30 and to more precisely control the extraction and retraction of the catcher wires 22 by operating the push handle member 10.

As a concrete embodiment shown in FIG. 1, each of the plurality of strands 20 may be formed in one body with each of catcher wires 22. By this embodiment, each of the strands of the inner core shaft 20 can be formed with a width equal to that of each of the catcher wires 22. It enables the hollow space for the guide wire 1 to be formed between the strands 20 as shown in FIG. 2(b) and the diameter of the outer sheath 30 to be minimized. Thus, it can be universally used to remove gallstones or calculi by using the endoscope or the percutaneous extraction, and foreign bodies from blood vessels and the like.

Here, the plurality of strands 20 may be consisted of 2-8 strands, but not restricted to the above numbers as long as it can play a role of the catcher wires 22 and be inserted into the outer sheath 30 having a predetermined inner diameter.

The catcher wires 22 and/or the plurality of strands 20 may be made of nitinol, platinum, titanium, stainless steel and the other elastic fibers. It is preferably to be made of shape memory alloy (SMA) changing into an original shape at a predetermined temperature (for example, at a body temperature) such as nitinol or super elastic alloy always returning to an original shape by removing the restraining force of the outer sheath 30.

Here, nitinol is an alloy of nickel and titanium with an atomic ratio 1:1 and changes into a preformed shape at a body temperature of 36° C. Thus, for using the above mentioned property, it is preferable that the catcher wires 22 and/or the plurality of strands 20 are made of nitinol.

Namely, nitinol has a property returning from any bent shape maintained in a body-centered cubic array at low temperature to an original shape having a face-centered cubic array by rearranging atoms located at the center of a cubic to each center of faces of a cubic at near body temperature.

Because the present invention use the property of shape memory alloy, such as nitinol, that returns to a preformed shape at a body temperature state by changing atomic array during the temperature change. It is not limed to nitinol but may be used as the shape memory alloy as long as having the above mentioned property such as Cu—Zn—Ni, Cu—Al—Ni, Ag—Ni, Au—Cd or the like combined between Ni, Cu, Fe or the like and another metal such as Zn, Al, Au, Ag or the like.

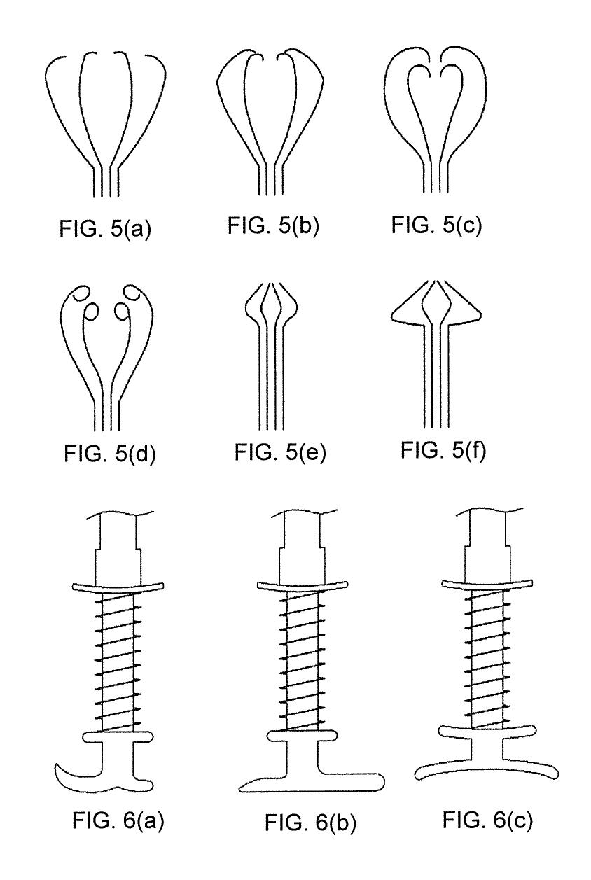

Therefore, if the catcher wires 22 and/or the plurality of strands 20 are formed of shape memory alloy, in order to return to a desired shape at a body temperature, the desired shapes of the catcher wires 22 and/or the plurality of strands 20 (for example, the catcher wires may have one of the shapes shown in FIG. 5 and the strands may have straight line shapes due to the placement within an outer sheath) are previously formed at the high temperature, and then, for occupying a minimal area within the outer sheath 30 at a temperature lower than the body temperature, the catcher wires 22 are flatted to straight line shapes as like as the strands 20 and installed to the stone extractors according to the present embodiment.

Particularly, when specific shapes of the catcher wires 22 of shape memory alloy are formed at high temperature such as 36° C. near to a body temperature, it is preferable that a distal end 24 of each of the catcher wires 22 is bending or curling toward the inside to form an atraumatic end as shown in FIG. 5 for preventing injuries on walls near the stones during operation.

FIG. 5 is showing exemplar shapes of the catcher wires 22 according to the present invention at a high temperature such as 36° C. near to a body temperature, but the shapes of the catcher wires 22 at the high temperature are not limited to the proposed examples.

In case that the catcher wires 22 are formed of super elastic alloy, when the catcher wires 22 are placed within the outer sheath 30, as shown in FIGS. 1(a) and 2(a), the catcher wires 22 are closely contacted and collapsed to the inside wall of the outer sheath 30 by the restraining force of the outer sheath 30 (this is the same state as the case that the catcher wires formed of shape memory alloy already reach the body temperature within the outer sheath) and when the push handle member 10 is distally pushed to press the elastic spring 40 as shown in FIG. 1(b), the catcher wires 22 are extracted from the outer sheath 30 and expanded to the original shapes. At this time, the catcher wires 22 capture adjacent stones.

Next, when the pushing force applied to the push handle member 10 is released, as shown in FIG. 1(c), the restoring force of the elastic spring 40 proximally pulls the catcher wires 22 through the push handle member 10 and the strands 20, and the distal end entrance of the catcher wires 22 is forced from a side wall of the distal end of the outer sheath 30 toward the inside thereof and then is shrunk and closed.

As mentioned above, because the distal end entrance of the catcher wires 22 is closed by the restoring force of the elastic spring 40, the captured stone is trapped in a cage formed by the catcher wires 22 or is bitten by the distal end entrance of the catcher wires 22 as shown in FIG. 10(c). Thus, the stone 5 can be moved to the exterior of a body in the bitten state and be efficiently removed.

On the other hand, the push handle member 10 can be configured to have not only a symmetric T-shaped handle as shown in FIG. 1, but also another shaped handle as shown in FIG. 6, such as an asymmetric T-shaped handle [FIG. 6(a)], an asymmetric H-shaped handle [FIG. 6(b)] or a symmetric H-shaped handle [FIG. 6(c)].

And the push handle member 10 may be further consisted of a fixing means to be fixed to a proximal end of the outer sheath 30 while the elastic spring 40 is pressed down. By this configuration, it can prevent the captured stone 5 or foreign body from being smashed and released unintentionally by the restoring force of the elastic spring 40.

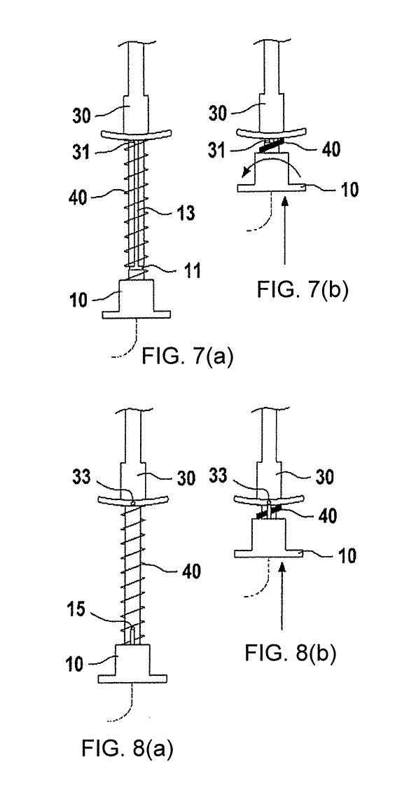

The fixing means can be embodied in various forms, but concrete embodiments shown in FIGS. 7 to 9 are described below.

According to an embodiment of FIG. 7, the push handle member 10, as shown in FIG. 1, may be consisted of a handle 12 and a supporting shaft 14 wrapped around by the elastic spring 40. The fixing means may be consisted of a first guide groove 13 formed at the supporting shaft 14 in a longitudinal direction and a second guide groove 11 formed at a proximal portion of the supporting shaft 14 in a circumference direction, the second guide groove 11 being vertically connected to the first guide groove 13. And the proximal end of the sheath 30 may have a groove inserting protrusion 31 configured to move along the first and second guide grooves 11 and 13.

By this configuration, as shown in FIG. 7(b), when the push handle member 10 is distally pushed to press the elastic spring 40 and rotated toward a left or right direction, the groove inserting protrusion 31 is slid along the first guide groove 13 and then inserted into the second guide groove 11 to intercept the restoring force of the elastic spring 40 and stop the push handle member 10 being proximally moved. Consequently, because the entrance of the distal end of the catcher wires 22 can constantly bite the stone 5 or the like and maintain the biting state, it can be prevented that the captured stone 5 or foreign body is smashed by the pressure of the entrance of the distal end of the catcher wires 22 due to the restoring force of the elastic spring 40.

According to an embodiment of FIG. 8, the fixing means may be consisted of a predetermined rack protrusion 15 connected to the handle of the push handle member 10 and the proximal end of the outer sheath 30 may have a concave groove 33 formed inside to be inserted by an end of the rack protrusion 15. Here, the push handle member 10 can further have a length-adjustment member (not shown) for adjusting a length of the rack protrusion 15.

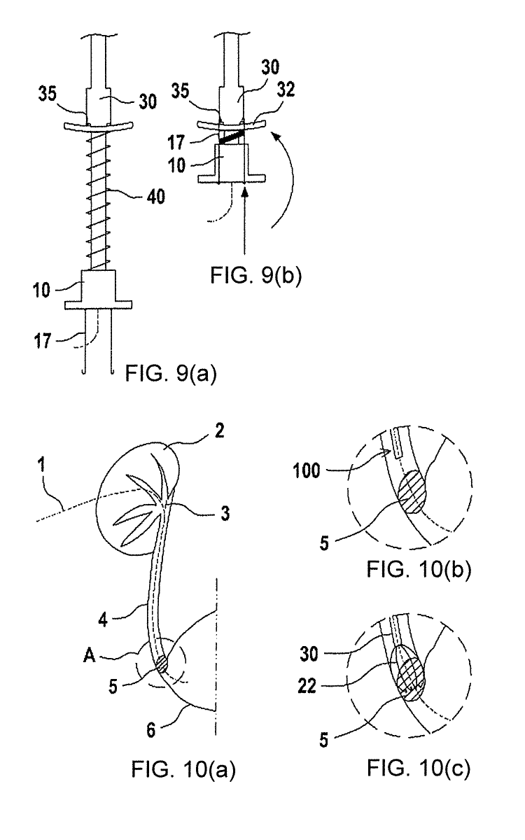

According to an embodiment of FIG. 9, the outer sheath 30 may be consisted of a body tube wrapping around the inner core shaft 20 and the catcher wires 22 and an entrance tube having a flange 32 connected to a proximal portion of the body tube. The fixing means may be consisted of one or more hook wires 17. One end of each of the hook wires 17 may be rotatably connected to the handle of the push handle member 10 and the other end of each of the hook wires 17 may have a bent shape to be hooked up to the flange 32 of the outer sheath 30. In FIG. 9, two of the hook wires 17 are shown, but is not limited to. In order to safely hook up the bending end each of the hook wires 17 to the flange 32, one or more corresponding protrusions 35 may be further formed on the flange 32. But they are not always needed.

FIG. 10 is an illustration and partially enlarged views of an ‘A’ part showing an example for removing a stone 5 in a urinary tract 4 by a stone extractor 100 according to the present invention.

As shown in FIG. 10(a), first, by the previous processes, a guide wire 1 is inserted from the pelvis 3 of the kidney 2 to pass by a stone, namely a calculus 5 in a urinary tract 4 and placed in a urinary bladder 6. The stone extractor 100 according to the present invention is inserted along the guide wire 1. When the distal end of the stone extractor 100 is located near to the stone 5 as shown in FIG. 10(b), the push handle member 10 is operated to extract the catcher wires 22 from the outer sheath 30 and to catch the stone 5 as shown in FIG. 10 (c). Then the pushing force applied to the push handle member 10 is cancelled, and the stone extractor 100 capturing the stone 5 is moved out the body along the guide wire 1 to remove the stone 5.

What is claimed is:

1. A stone extractor comprising:
an inner core shaft formed of a single or plurality of strands, the inner core shaft having a hollow space in a longitudinal direction for inserting a guide wire;
catcher wires connected to a distal end of the inner core shaft;
a push handle member connected to a proximal end of the inner core shaft, the push handle member having a penetrating hole in the middle along a longitudinal direction;
a flexible outer sheath configured to wholly wrap around the inner core shaft and the catcher wires and to extract and retract the catcher wires by operating the push handle member; and
an elastic spring installed between a proximal end of the flexible outer sheath and the push handle member.

2. The stone extractor of claim 1,
wherein the inner core shaft is formed of a single strand having a penetrating hole in the middle along a longitudinal direction as the hollow space.

3. The stone extractor of claim 2,
wherein the single strand is formed in one body with the push handle member.

4. The stone extractor of claim 1,
wherein the inner core shaft is formed of a plurality of strands,
wherein the hollow space is disposed between the plurality of strands, and
wherein each of the catcher wires is connected to each of the plurality of strands in one-to-one correspondence.

5. The stone extractor of claim 4,
wherein the plurality of strands are tied by one or more stabilizing members along a longitudinal direction.

6. The stone extractor of claim 4,
wherein each of the plurality of strands is formed in one body with each of the catcher wires.

7. The stone extractor of claim 6,
wherein the plurality of strands are consisted of 2˜8 strands.

8. The stone extractor of claim 1,
wherein the catcher wires are made of shape memory alloy changing into a preformed shape at a predetermined temperature or super elastic alloy returning to an original shape by removing the restraining force of the outer sheath.

9. The stone extractor of claim 8,
wherein each of the catcher wires has an atraumatic distal end bending or curling toward the inside of the catcher wires.

10. The stone extractor of claim 8,
wherein the push handle member has one of asymmetric T, symmetric T, asymmetric H and symmetric H-shaped handles.

11. The stone extractor of claim 8,
wherein the push handle member further has a fixing means to be fixed to a proximal end of the outer sheath while the elastic spring is pressed down.

12. The stone extractor of claim 11,
wherein the push handle member further comprises a supporting shaft to be wrapped around by the elastic spring, the supporting shaft being disposed between the handle and the inner core shaft,
wherein the fixing means is consisted of a first guide groove formed at the supporting shaft in a longitudinal direction and a second guide groove formed at a proximal portion of the supporting shaft in a circumference direction, the second guide groove being vertically connected to the first guide groove, and
wherein the proximal end of the sheath has a groove inserting protrusion configured to move along the first and second guide grooves.

13. The stone extractor of claim 11,
wherein the fixing means is consisted of a rack protrusion connected to the handle, and
wherein the proximal end of the outer sheath has a concave grooves formed inside to be inserted by the rack protrusion.

14. The stone extractor of claim 11,
wherein the outer sheath is consisted of a body tube wrapping around the inner core shaft and the catcher wires and an entrance tube having a flange connected to a proximal portion of the body tube,
wherein the fixing means is consisted of one or more hook wires, and
wherein one end of each of the hook wires is rotatably connected to the handle and the other end of each of the hook wires has a bent shape to be hooked up to the flange of the outer sheath.

15. The stone extractor of claim 4,
wherein the catcher wires are made of shape memory alloy changing into an original shape at a predetermined temperature or super elastic alloy returning to an original shape by removing the restraining force of the outer sheath.

16. The stone extractor of claim 15,
wherein each of the catcher wires has an atraumatic distal end bending or curling toward the inside of the catcher wires.

17. The stone extractor of claim 15,
wherein the push handle member further has a fixing means to be fixed to a proximal end of the outer sheath while the elastic spring is pressed down.

18. The stone extractor of claim 17,
wherein the push handle member further comprises a supporting shaft to be wrapped around by the elastic spring, the supporting shaft being disposed between the handle and the inner core shaft,
wherein the fixing means is consisted of a first guide groove formed at the supporting shaft in a longitudinal direction and a second guide groove formed at a proximal portion of the supporting shaft in a circumference direction, the second guide groove being vertically connected to the first guide groove, and
wherein the proximal end of the sheath has a groove inserting protrusion configured to move along the first and second guide grooves.

19. The stone extractor of claim 17,
wherein the fixing means is consisted of a rack protrusion connected to the handle, and
wherein the proximal end of the outer sheath has a concave grooves formed inside to be inserted by the rack protrusion.

20. The stone extractor of claim 17,
wherein the outer sheath is consisted of a body tube wrapping around the inner core shaft and the catcher wires and an entrance tube having a flange connected to a proximal portion of the body tube,
wherein the fixing means is consisted of one or more hook wires, and
wherein one end of each of the hook wires is rotatably connected to the handle and the other end of each of the hook wires has a bent shape to be hooked up to the flange of the outer sheath.
